(19) United States
(12) Patent Application Publication
(10) Pub. No.: US 2013/0210885 A1
(43) Pub. Date: Aug. 15, 2013

(54) CRYSTALLINE FORMS OF L-MALIC ACID SALT OF SUNITINIB

(71) Applicants: Sudhir Singh Sanwal, Kangra (IN); Saridi Madhava Dileep Kumar, Gurgaon (IN); Swargam Sathyanarayana, Karim Nagar (IN); Rajesh Kumar Thaper, Jammu (IN); Mohan Prasad, Gurgaon (IN)

(72) Inventors: Sudhir Singh Sanwal, Kangra (IN); Saridi Madhava Dileep Kumar, Gurgaon (IN); Swargam Sathyanarayana, Karim Nagar (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/575,424

(22) PCT Filed: Jan. 28, 2011
(86) PCT No.: PCT/IB2011/050397

§ 371 Date: Apr. 11, 2013

(30) Foreign Application Priority Data
Jan. 29, 2010 (IN) 203/DEL/2010

(51) Int. Cl. C07D 403/06

(52) U.S. Cl. CPC C07D 403/06
USPC 514/414; 548/468

(57) ABSTRACT

The present invention relates to crystalline forms of L-malic acid salt of sunitinib and its preparation. The crystalline forms of the present invention are designated as Form V and Form VI of L-malic acid salt of sunitinib. Formula (I).

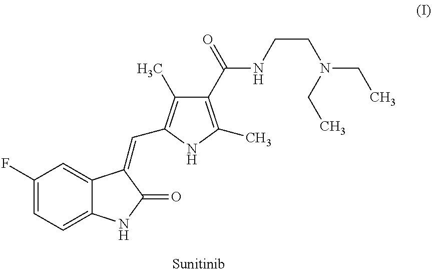 [SMILES: CCN(CC)CCNC(=O)c1c(C)[nH]c(/C=C2\C(=O)Nc3ccc(F)cc32)c1C]

### FIELD OF THE INVENTION

The present invention relates to crystalline forms of L-malic acid salt of sunitinib and processes for their preparation. The crystalline forms of the present invention are designated as Form V and Form VI of L-malic acid salt of sunitinib.

### BACKGROUND OF THE INVENTION

Sunitinib is chemically described as N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide as represented by Formula I.

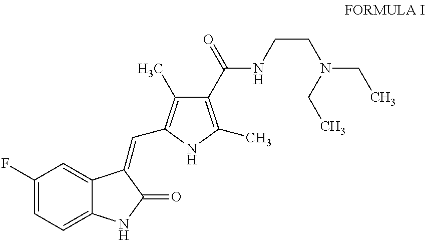 [SMILES: CCN(CC)CCNC(=O)c1c(C)[nH]c(/C=C2\C(=O)Nc3ccc(F)cc32)c1C]

Sunitinib is an oral multi-kinase inhibitor and useful for the treatment of gastrointestinal stromal tumor and advanced renal cell carcinoma. Sunitinib is commercially available as a L-malate salt, which is described chemically as butanedioic acid, hydroxy-, (2S)-, compound with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1).

U.S. Publication Nos. 2003/0069298 and 2007/0191458 describe the preparation of crystal Forms I and II of L-malic acid salt of sunitinib. According to the above publications, crystal Form I of L-malic acid salt of sunitinib is prepared by slurrying a poorly crystalline or crystal Form II of L-malic acid salt of sunitinib in acetonitrile. Crystal Form II of L-malic acid salt of sunitinib is prepared by dissolving crystal Form I of L-malic acid salt of sunitinib in tetrahydrofuran and water and allowing the solvent to evaporate overnight. WO 2009/067686 describes processes for preparing crystalline forms of racemic sunitinib malate, sunitinib hemi-L-malate and compositions containing sunitinib base and L- or racemic malic acid.

WO 2009/104021 describes processes for preparing crystalline Forms III and IV of sunitinib malate. WO 2009/128083 describes processes for preparing crystalline Forms A, B, C, D, E, F and G of sunitinib base. WO 09/109,388 describes processes for preparing crystalline Forms I, II and III of sunitinib base. WO 2009/156837 describes processes for preparing amorphous form of L-malic acid salt of sunitinib. WO 2010/004339 describes processes for preparing crystalline Form I of sunitinib malate.

### SUMMARY OF THE INVENTION

A crystalline Form V of L-malic acid salt of sunitinib.

A crystalline Form V of L-malic acid salt of sunitinib comprising an XRPD as depicted in FIG. 1.

A crystalline Form V of L-malic acid salt of sunitinib which is characterized by an XRPD pattern comprising interplanar spacing (d) values substantially at 6.51, 5.99, 5.83, 5.02, 4.76, 4.63, 3.85, 3.82, 3.64, 3.60, 3.56 and 3.09±0.02 Å.

A crystalline Form V of L-malic acid salt of sunitinib which is further characterized by an XRPD pattern further comprising interplanar spacing (d) values substantially at 15.42, 11.68, 11.02, 10.05, 9.25, 7.97, 7.65, 6.97, 6.83, 6.73, 6.51, 5.99, 5.83, 5.66, 5.56, 5.50, 5.29, 5.25, 5.15, 5.02, 4.82, 4.76, 4.63, 4.58, 4.50, 4.35, 4.28, 4.19, 4.10, 4.06, 4.00, 3.95, 3.84, 3.82, 3.76, 3.68, 3.64, 3.60, 3.56, 3.49, 3.41, 3.39, 3.31, 3.24, 3.17, 3.09, 3.04, 2.99, 2.95, 2.91, 2.88, 2.85, 2.79, 2.76, 2.72, 2.66, 2.56, 2.52, 2.47, 2.41, 2.35 and 2.31±0.02 Å.

A crystalline Form V of L-malic acid salt of sunitinib comprising a dimethylsulfoxide content from about 7.5% to about 10.5%.

A crystalline Form V of L-malic acid salt of sunitinib according to the claim 5, comprising a dimethylsulfoxide content from about 8.5% to about 9.5%.

A crystalline Form VI of L-malic acid salt of sunitinib.

A crystalline Form VI of L-malic acid salt of sunitinib comprising an XRPD as depicted in FIGS. 2 and 3.

A crystalline Form VI of L-malic acid salt of sunitinib which is characterized by an XRPD pattern comprising interplanar spacing (d) values substantially at 15.37, 7.42, 6.76, 6.40, 6.22, 5.58, 5.49, 5.06, 4.82, 4.58, 3.93, 3.68, 3.49, 3.46, 3.41, 3.35, 3.23 and 3.11±0.02 Å.

A crystalline Form VI of L-malic acid salt of sunitinib which is further characterized by an XRPD pattern comprising interplanar spacing (d) values substantially at 27.72, 15.37, 9.22, 7.68, 7.42, 6.76, 6.39, 6.22, 5.58, 5.49, 5.29, 5.24, 5.15, 5.06, 4.97, 4.82, 4.70, 4.58, 4.48, 4.37, 4.23, 4.19, 4.10, 4.00, 3.93, 3.84, 3.77, 3.68, 3.49, 3.46, 3.41, 3.35, 3.23, 3.11, 3.07, 2.98, 2.92, 2.85, 2.80, 2.72, 2.6 3, 2.57, 2.52, 2.46, 2.44, 2.41, 2.35 and 2.30±0.02 Å.

A process for the preparation of crystalline Form V of L-malic acid salt of sunitinib, wherein the process includes:
a) treating L-malic acid salt of sunitinib with dimethylsulfoxide; and
b) isolating the crystalline Form V of L-malic acid salt of sunitinib from the mixture thereof.

A process according to the claim 11, wherein the treatment with dimethylsulfoxide is carried out at a temperature of about 50° C. to about 75° C.

A process according to the claim 12, wherein the treatment with dimethylsulfoxide is carried out at a temperature of about 65° C. to about 70° C.

A process according to the claim 11, wherein the crystalline Form V of L-malic acid salt of sunitinib is isolated by stirring.

A process according to the claim 14, wherein the crystalline Form V of L-malic acid salt of sunitinib is isolated at a temperature of about 30° C. or less.

A process for the preparation of crystalline Form VI of L-malic acid salt of sunitinib, wherein the process includes:
a) treating crystalline Form V of L-malic acid salt of sunitinib with an ester or an alcohol; and
b) isolating crystalline Form VI of L-malic acid salt of sunitinib.

A process according to the claim 16, wherein the ester comprises methyl acetate.

A process according to the claim 16, wherein the alcohol comprises methanol.

A process according to the claim 16, wherein the treatment with an ester or an alcohol is carried out at a temperature of about 10° C. to about 50° C.

A process according to the claim 16, wherein the treatment with an ester or an alcohol is carried out at a temperature of about 15° C. to about 30° C.

A pharmaceutical composition comprising crystalline Form VI of L-malic acid salt of sunitinib and a carrier.

A method of treating or preventing a protein kinase related disorder comprising administering to a patient in need thereof a therapeutically effective amount of a crystalline Form VI of L-malic acid salt of sunitinib.

### BRIEF DESCRIPTION OF THE DRAWINGS

FIG. 1 depicts the X-ray powder diffraction pattern (XRPD) of the crystalline Form V of L-malic acid salt of sunitinib.

FIG. 1A provides the table of values for the XRPD pattern depicted in FIG. 1.

FIG. 2 depicts the X-ray powder diffraction pattern (XRPD) of the crystalline Form VI of L-malic acid salt of sunitinib obtained according to Examples 2.

FIG. 2A provides the table of values for the XRPD pattern depicted in FIG. 2.

FIG. 3 depicts the X-ray powder diffraction pattern (XRPD) of the crystalline Form VI of L-malic acid salt of sunitinib obtained according to Examples 3.

FIG. 3A provides the table of values for the XRPD pattern depicted in FIG. 3.

### DETAILED DESCRIPTION OF THE INVENTION

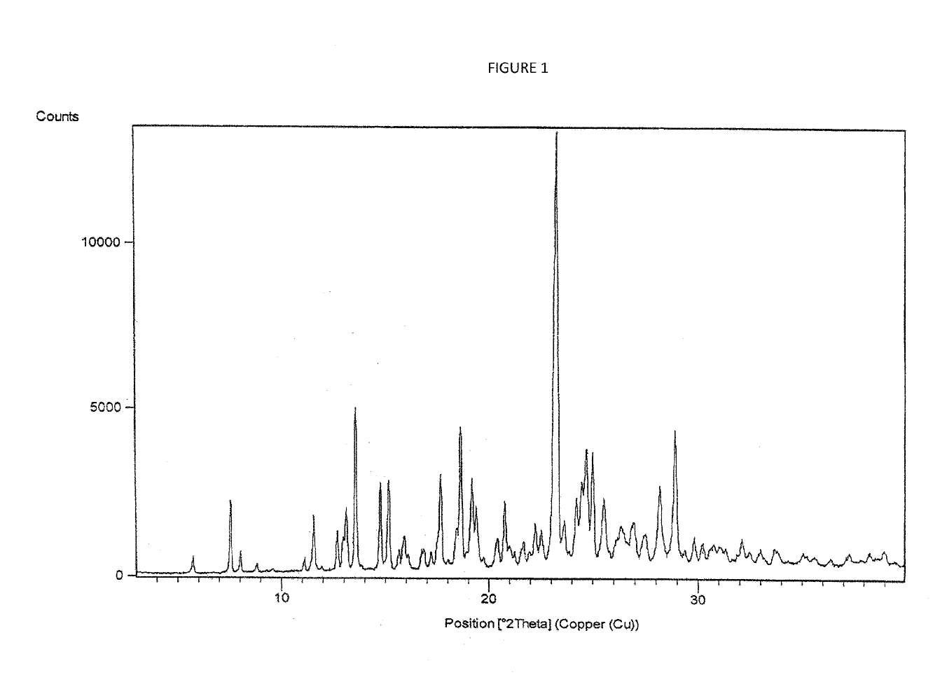

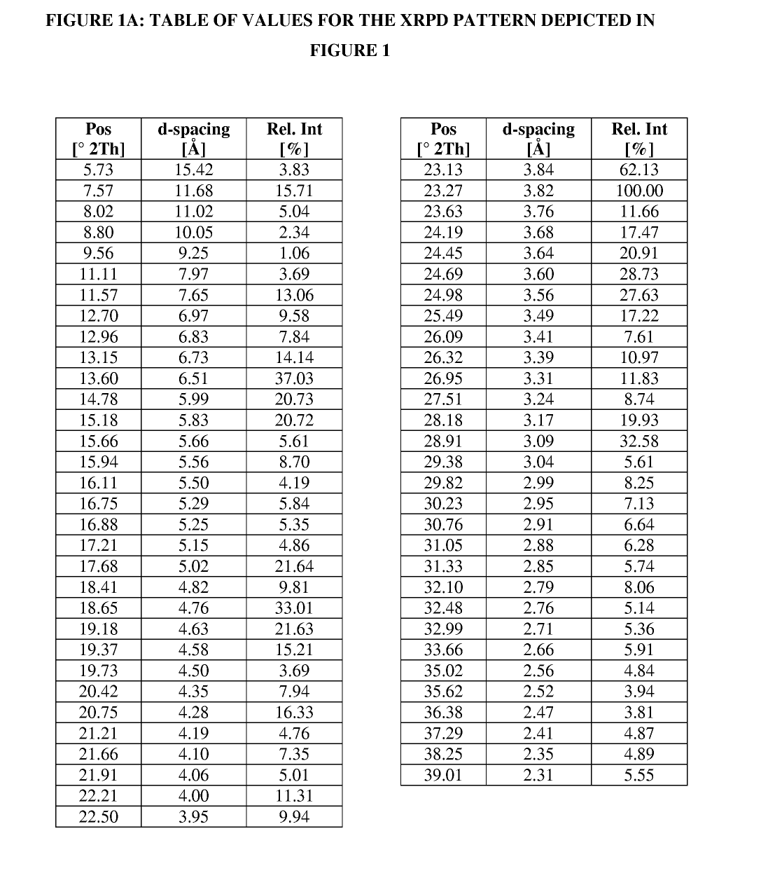

The present invention provides for crystalline Form V of L-malic acid salt of sunitinib. The crystalline Form V of L-malic acid salt of sunitinib has substantially the same XRPD (X-ray powder diffraction pattern) pattern as depicted in FIG. 1. The crystalline Form V of L-malic acid salt of sunitinib is characterized by an XRPD pattern which includes interplanar spacing (d) values substantially at 6.51, 5.99, 5.83, 5.02, 4.76, 4.63, 3.85, 3.82, 3.64, 3.60, 3.56 and 3.09±0.02 Å. The crystalline Form V of L-malic acid salt of sunitinib is further characterized by an XRPD pattern that includes interplanar spacing (d) values substantially at 15.42, 11.68, 11.02, 10.05, 9.25, 7.97, 7.65, 6.97, 6.83, 6.73, 6.51, 5.99, 5.83, 5.66, 5.56, 5.50, 5.29, 5.25, 5.15, 5.02, 4.82, 4.76, 4.63, 4.58, 4.50, 4.35, 4.28, 4.19, 4.10, 4.06, 4.00, 3.95, 3.84, 3.82, 3.76, 3.68, 3.64, 3.60, 3.56, 3.49, 3.41, 3.39, 3.31, 3.24, 3.17, 3.09, 3.04, 2.99, 2.95, 2.91, 2.88, 2.85, 2.79, 2.76, 2.72, 2.66, 2.56, 2.52, 2.47, 2.41, 2.35 and 2.31±0.02 Å.

The crystalline Form V of L-malic acid salt of sunitinib has a dimethylsulfoxide content from about 7.5% to about 10.5%, for example, from about 8.5% to about 9.5%.

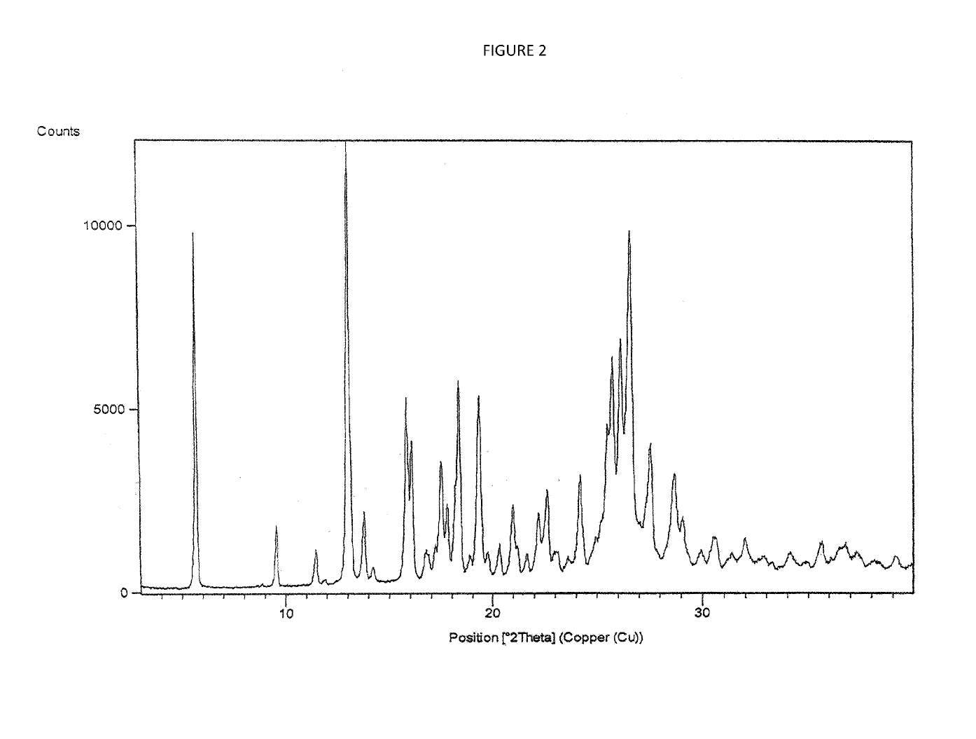

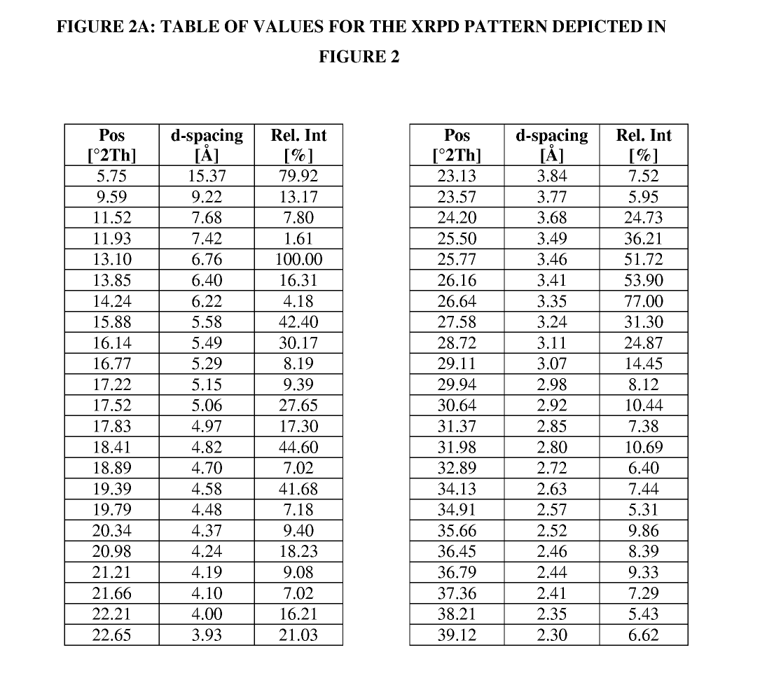

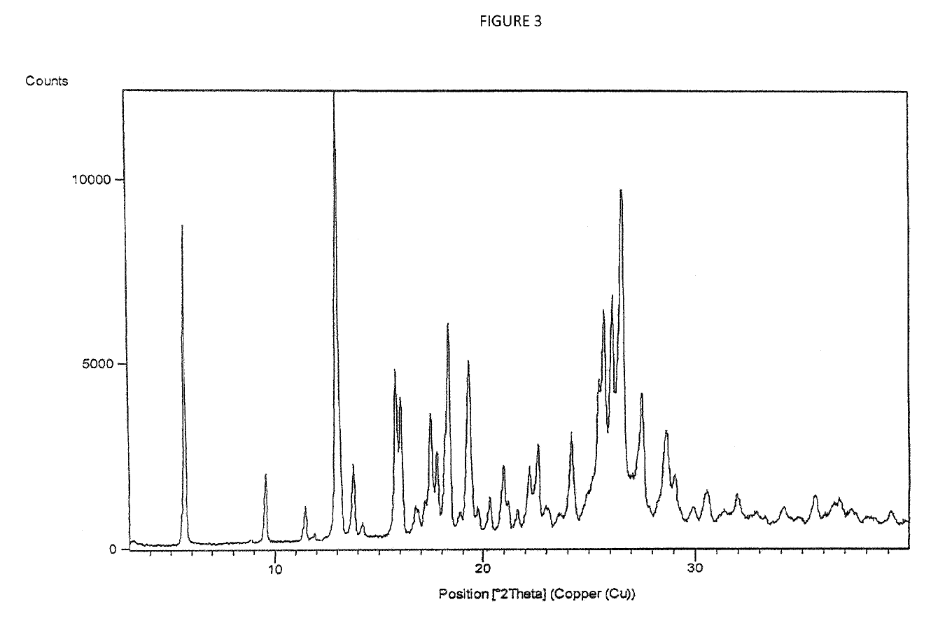

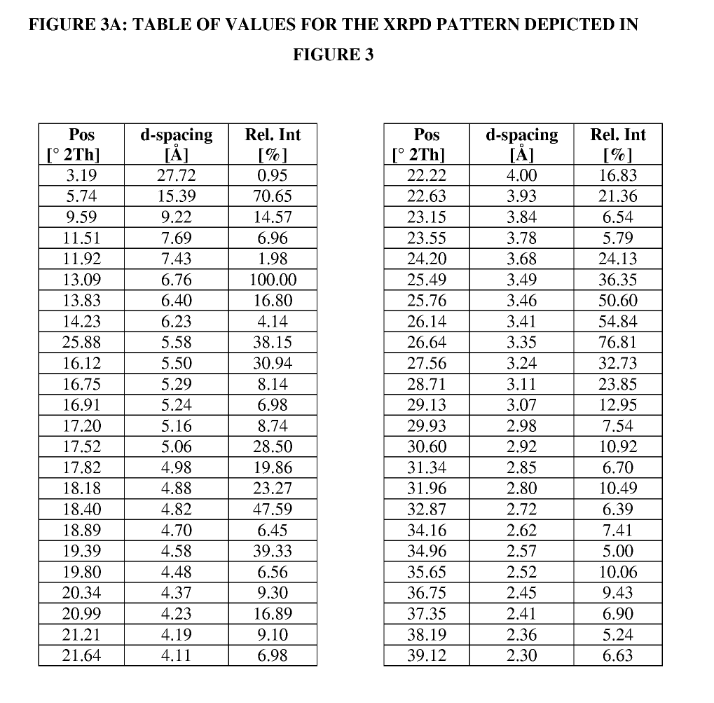

The present invention also provides for crystalline Form VI of L-malic acid salt of sunitinib. The crystalline Form VI of L-malic acid salt of sunitinib has substantially the same XRPD (X-ray powder diffraction pattern) pattern as depicted in FIGS. 2 and 3. The crystalline Form VI of L-malic acid salt of sunitinib is characterized by an XRPD pattern that includes interplanar spacing (d) values substantially at 15.37, 7.42, 6.76, 6.40, 6.22, 5.58, 5.49, 5.06, 4.82, 4.58, 3.93, 3.68, 3.49, 3.46, 3.41, 3.35, 3.23 and 3.11±0.02 Å. The crystalline Form VI of L-malic acid salt of sunitinib is further characterized by an XRPD pattern that includes interplanar spacing (d) values substantially at 27.72, 15.37, 9.22, 7.68, 7.42, 6.76, 6.39, 6.22, 5.58, 5.49, 5.29, 5.24, 5.15, 5.06, 4.97, 4.82, 4.70, 4.58, 4.48, 4.37, 4.23, 4.19, 4.10, 4.00, 3.93, 3.84, 3.77, 3.68, 3.49, 3.46, 3.41, 3.35, 3.23, 3.11, 3.07, 2.98, 2.92, 2.85, 2.80, 2.72, 2.6 3, 2.57, 2.52, 2.46, 2.44, 2.41, 2.35 and 2.30±0.02 Å.

The present invention also provides for a process for the preparation of crystalline Form V of L-malic acid salt of sunitinib. The process includes:
a) treating L-malic acid salt of sunitinib with dimethylsulfoxide; and
b) isolating the crystalline Form V of L-malic acid salt of sunitinib from the mixture thereof.

L-Malic acid salt of sunitinib existing in any solid form known in the prior art may be used as the starting material. The L-malic acid salt of sunitinib may be prepared according to the methods disclosed in Indian Patent Nos. 2337/DEL/2009, and 2386/DEL/2009. The L-malic acid salt of sunitinib is treated with dimethylsulfoxide. The treatment with dimethylsulfoxide may be carried out at a temperature of about 50° C. to about 75° C., for example, from about 65° C. to about 70° C., to obtain a solution. The treatment with dimethylsulfoxide may be accompanied by stiffing. The crystalline Form V of L-malic acid salt of sunitinib is isolated, for example, by stirring at a temperature of about 30° C. or less, for example, for about 15° C. to about 25° C. The stiffing may be carried out for about 30 minutes to about 48 hours, for example, about 6 hours to about 15 hours. The excess of dimethylsulfoxide, if any, may be removed by filtration, distillation, decantation, vacuum drying, evaporation, or a combination thereof, to obtain the crystalline Form V of L-malic acid salt of sunitinib. The crystalline Form V of L-malic acid salt of sunitinib has dimethylsulfoxide content from about 7.5% to about 10.5%, for example, from about 8.5% to about 9.5%.

The present invention also provides for a process for the preparation of crystalline Form VI of L-malic acid salt of sunitinib. The process includes:
a) treating crystalline Form V of L-malic acid salt of sunitinib with an ester or an alcohol; and
b) isolating crystalline Form VI of L-malic acid salt of sunitinib.

The crystalline Form V of L-malic acid of sunitinib may be prepared according to the previous aspect of the present invention. The crystalline Form V of L-malic acid salt of sunitinib is treated with an ester or an alcohol. The ester may be, for example, methyl acetate and the alkanol may be, for example, methanol, or a mixture thereof. The treatment with the solvent may be carried out at a temperature of about 10° C. to about 50° C., for example, about 15° C. to about 30° C. accompanied by stirring. The stirring may be carried out for about 1 hour to about 50 hours, for example, about 3 hours to 10 hours. The crystalline Form VI of L-malic acid salt of sunitinib may be isolated by filtration, distillation, decantation, vacuum drying, evaporation, or a combination thereof.

The present invention also provides for a pharmaceutical composition that includes crystalline Form VI of L-malic acid salt of sunitinib and a carrier.

The present invention also provides for a method of treating or preventing a protein kinase related disorder, which includes administering to a patient in need thereof a therapeutically effective amount of a crystalline Form VI of L-malic acid salt of sunitinib. The XRPD of the samples were determined by using Panalytical X′Pert Pro X-Ray Powder Diffractometer in the range 3-40 degree 2 theta and under tube voltage and current of 45 Kv and 40 mA, respectively. Copper radiation of wavelength 1.54 angstrom and Xceletor detector was used.

While the present invention has been described in terms of its specific embodiments, certain modifications and equivalents will be apparent to those skilled in the art and are intended to be included within the scope of the present invention.

### EXAMPLES

### Example 1

### Preparation of Crystalline Form V of L-Malic Acid Salt of Sunitinib

L-Malic acid salt of sunitinib (20 g) was dissolved in dimethylsulfoxide (80 ml) by stirring at 65° C. to 70° C. for 30 minutes. The solution was slowly cooled to 20° C. to 25° C. in 1 hour and stirred at 20° C. to 25° C. for 15 hours. The mixture was filtered under vacuum at 20° C. to 25° C. and the solid was dried under vacuum at 60° C. to 65° C. for 24 hours to obtain the title compound.

### Yield: 14.5 g

Dimethylsulfoxide content: 9.07% (by thermo gravimetric analysis)

### Example 2

### Preparation of Crystalline Form VI of L-Malic Acid Salt of Sunitinib

Crystalline Form V of L-malic acid salt of sunitinib (2 g) was stirred in methanol (12 ml) at 20° C. to 22° C. for 5 hours to 6 hours. The mixture was filtered under vacuum at 20° C. to 25° C. and dried under vacuum at 60° C. for 12 hours to 15 hours to obtain the title compound.

### Yield: 1.5 g

### Example 3

### Preparation of Crystalline Form VI of L-Malic Acid Salt of Sunitinib

Crystalline Form V of L-malic acid salt of sunitinib (2 g) was stirred in methyl acetate (12 ml) at 20° C. to 22° C. for 5 hours to 6 hours. The mixture was filtered under vacuum at 20° C. to 25° C. and dried under vacuum at 60° C. for 12 hours to 15 hours to obtain the title compound.

Yield: 1.5 g

1. (canceled)

2. A crystalline Form V of L-malic acid salt of sunitinib having substantially the same XRPD (X-ray powder diffraction pattern) pattern as depicted in FIG. 1.

3. A crystalline Form V of L-malic acid salt of sunitinib which is characterized by an XRPD pattern comprising interplanar spacing (d) values substantially at 6.51, 5.99, 5.83, 5.02, 4.76, 4.63, 3.85, 3.82, 3.64, 3.60, 3.56 and 3.09±0.02 Å.

4. A crystalline Form V of L-malic acid salt of sunitinib according to the claim 3 which is characterized by an XRPD pattern comprising interplanar spacing (d) values substantially at 15.42, 11.68, 11.02, 10.05, 9.25, 7.97, 7.65, 6.97, 6.83, 6.73, 6.51, 5.99, 5.83, 5.66, 5.56, 5.50, 5.29, 5.25, 5.15, 5.02, 4.82, 4.76, 4.63, 4.58, 4.50, 4.35, 4.28, 4.19, 4.10, 4.06, 4.00, 3.95, 3.84, 3.82, 3.76, 3.68, 3.64, 3.60, 3.56, 3.49, 3.41, 3.39, 3.31, 3.24, 3.17, 3.09, 3.04, 2.99, 2.95, 2.91, 2.88, 2.85, 2.79, 2.76, 2.72, 2.66, 2.56, 2.52, 2.47, 2.41, 2.35 and 2.31±0.02 Å.

5. A crystalline Form V of L-malic acid salt of sunitinib having interplanar spacing (d) values in XRPD substantially at 6.51, 5.99, 5.83, 5.02, 4.76, 4.63, 3.85, 3.82, 3.64, 3.60, 3.56 and 3.09±0.02 Å comprising a dimethylsulfoxide content from about 7.5% to about 10.5%.

6. (canceled)

7. (canceled)

8. A crystalline Form VI of L-malic acid salt of sunitinib having substantially the same XRPD (X-ray powder diffraction pattern) pattern as depicted in FIGS. 2 and 3.

9. A crystalline Form VI of L-malic acid salt of sunitinib which is characterized by an XRPD pattern comprising interplanar spacing (d) values substantially at 15.37, 7.42, 6.76, 6.40, 6.22, 5.58, 5.49, 5.06, 4.82, 4.58, 3.93, 3.68, 3.49, 3.46, 3.41, 3.35, 3.23 and 3.11±0.02 Å.

10. A crystalline Form VI of L-malic acid salt of sunitinib according to the claim 9 which is characterized by an XRPD pattern comprising interplanar spacing (d) values substantially at 27.72, 15.37, 9.22, 7.68, 7.42, 6.76, 6.39, 6.22, 5.58, 5.49, 5.29, 5.24, 5.15, 5.06, 4.97, 4.82, 4.70, 4.58, 4.48, 4.37, 4.23, 4.19, 4.10, 4.00, 3.93, 3.84, 3.77, 3.68, 3.49, 3.46, 3.41, 3.35, 3.23, 3.11, 3.07, 2.98, 2.92, 2.85, 2.80, 2.72, 2.6 3, 2.57, 2.52, 2.46, 2.44, 2.41, 2.35 and 2.30±0.02 Å.

11. A process for the preparation of crystalline Form V of L-malic acid salt of sunitinib having interplanar spacing (d) values in XRPD substantially at 6.51, 5.99, 5.83, 5.02, 4.76, 4.63, 3.85, 3.82, 3.64, 3.60, 3.56 and 3.09±0.02 according to claim 3, wherein the process includes:
a) treating L-malic acid salt of sunitinib with dimethylsulfoxide; and
b) isolating the crystalline Form V of L-malic acid salt of sunitinib from the mixture thereof.

12. A process according to the claim 11, wherein the treatment with dimethylsulfoxide is carried out at a temperature of about 50° C. to about 75° C.

13. (canceled)

14. A process according to the claim 11, wherein the crystalline Form V of L-malic acid salt of sunitinib is isolated by stirring.

15. A process according to the claim 14, wherein the crystalline Form V of L-malic acid salt of sunitinib is isolated at a temperature of about 30° C. or less.

16. A process for the preparation of crystalline Form VI of L-malic acid salt of sunitinib having interplanar spacing (d) values in XRPD substantially at 15.37, 7.42, 6.76, 6.40, 6.22, 5.58, 5.49, 5.06, 4.82, 4.58, 3.93, 3.68, 3.49, 3.46, 3.41, 3.35, 3.23 and 3.11±0.02 Å according to claim 9, wherein the process includes:
a) treating crystalline L-malic acid salt of sunitinib having interplanar spacing (d) values in XRPD substantially at 6.51, 5.99, 5.83, 5.02, 4.76, 4.63, 3.85, 3.82, 3.64, 3.60, 3.56 and 3.09±0.02 with an ester or an alcohol; and
b) isolating crystalline Form VI of L-malic acid salt of sunitinib.

17. A process according to the claim 16, wherein the ester comprises methyl acetate.

18. A process according to the claim 16, wherein the alcohol comprises methanol.

19. A process according to the claim 16, wherein the treatment with an ester or an alcohol is carried out at a temperature of about 10° C. to about 50° C.

20. (canceled)

21. A pharmaceutical composition comprising crystalline Form VI having interplanar spacing (d) values in XRPD substantially at 15.37, 7.42, 6.76, 6.40, 6.22, 5.58, 5.49, 5.06, 4.82, 4.58, 3.93, 3.68, 3.49, 3.46, 3.41, 3.35, 3.23 and 3.11±0.02 Å of L-malic acid salt of sunitinib according to claim 9 and a carrier.

22. A method of treating or preventing a protein kinase related disorder comprising administering to a patient in need thereof a therapeutically effective amount of a crystalline Form VI of L-malic acid salt of sunitinib according to claim 9 having interplanar spacing (d) values in XRPD substantially at 15.37, 7.42, 6.76, 6.40, 6.22, 5.58, 5.49, 5.06, 4.82, 4.58, 3.93, 3.68, 3.49, 3.46, 3.41, 3.35, 3.23 and 3.11±0.02 Å.
